(19) United States
(12) Patent Application Publication
(10) Pub. No.: US 2012/0294806 A1
(43) Pub. Date: Nov. 22, 2012

(54) DRUG CARRIER WITH THERMAL SENSITIVITY, MANUFACTURING METHOD THEREOF, AND USE THEREOF

(76) Inventors: San-Yuan CHEN, Hsinchu City (TW); Shang-Hsiu HU, Taoyuan County (TW); Hsin-Yang HUANG, Kaohsiung City (TW)

(21) Appl. No.: 13/281,104

(22) Filed: Oct. 25, 2011

(30) Foreign Application Priority Data
May 17, 2011 (TW) 100117136

(51) Int. Cl. A61K 49/18; A61P 35/00; A61K 9/14

(52) U.S. Cl. 424/9.3; 424/490; 424/489; 977/773

(57) ABSTRACT

A drug carrier with thermal sensitivity, a manufacturing method thereof, and a use thereof are disclosed. The drug carrier comprises a nano-magnetic particle, a drug, a composite polymer, and a dense silica shell. The nano-magnetic particle and the drug are encapsulated in the composite polymer which is formed by self-assembly a water-soluble polymer (such as poly vinyl alcohol) and a thermosensitive copolymer (such as Pluronic F68 or Pluronic F127). The stability and drug release of the drug carrier a can be adjusted by combining PVA and thermosensitive copolymer with a different ratio. When an external magnetic field was applied, the cores exhibit significant size shrinkage and the diameter of the drug carrier decreases more than 10 folds due to the change of temperature, which causes burst-like drug release because of shell destruction and physical collapse of the drug carrier.

### CROSS-REFERENCE TO RELATED APPLICATION

This application claims priority from Taiwan Patent Application No .100117136, filed on May 17, 2011, in Taiwan Intellectual Property Office, the contents of which are hereby incorporated by reference in their entirety for all purposes.

### BACKGROUND OF THE INVENTION

1. Field of the Invention

The present invention relates to a drug carrier and a manufacturing method and a use thereof, in particular to a magnetically controlled drug carrier with thermal sensitivity, a manufacturing method thereof, and a use of the same as a NMR contrast agent.

2. Description of the Related Art

In recent years, the cancer rate in every country increases every year, so that how to design and control a drug release system for a target treatment of cancer becomes significantly important. However, most of the conventional drug release control systems are composed of acids, alkalis and thermosensitive materials. The conventional drug control systems only have effects in certain parts of human body so as to fail to fit different situations of controlling the drug release.

Since the magnetic field is associated with force at a distance, the magnetic field can be applied externally to control the drug release system to release drug, so as to perform a local treatment at any position of a human body. In addition, the magnetic-control drug release system includes magnetic particles, and magnetic resonance imaging (MRI) can be used for tracking or positioning to release the drug to a specific position in order to improve the local treatment effect significantly.

However, most of the drug release systems or drug carriers have loosened and unstable structures. When the conventional drug carriers as disclosed in patents such as WO2010134087, US20090324494, US20050130167, and CN200310122436 are applied to human bodies, the structures thereof will be damaged very easily, and the drug may be leaked easily during the drug delivery process. Thus, a specific drug cannot be released to a specific position. Particularly, the thermosensitive polymers have the property of a lower critical solution temperature (LCST). If a nano drug carrier made of the thermosensitive polymers is produced without using a chemical crosslinking agent, the thermosensitive nano drug carrier will have an unstable structure and incur a difficult manufacture. On the other hand, a use of chemical crosslinking agents usually gives a poor biocompatibility.

Therefore, it is a primary issue to prepare a stable thermosensitive nano drug carrier effectively without using any chemical crosslinking agent and achieve the effects of reducing a natural drug leakage before the drug carrier moves to a target position, and controlling the drug release by applying an external magnetic field after the drug carrier has moved to the target position.

### SUMMARY OF THE INVENTION

In view of the shortcomings of the prior art, it is a primary objective of the present invention to provide a drug carrier with high biocompatibility and thermosensitivity circulated in human body for a long time and the effect of a highly sensitive NMR contrast agent. Meanwhile, the manufacturing method thereof does not require any chemical crosslinking agent and can prepare a stable drug carrier by a simple and easy manufacturing process and reduce the natural drug leakage to a level approaching zero during a drug delivery. Therefore, the problem of the conventional drug release system failing to control the local release of drug via external energy can be overcome.

To achieve the aforementioned objective, the present invention provides a drug carrier, comprising a nano-magnetic particle, a drug and a composite polymer. The nano-magnetic particle and the drug are encapsulated in the composite polymer. The composite polymer is made of a water-soluble polymer (such as poly vinyl alcohol, PVA) and a thermosensitive copolymer (such as poly(ethylene oxide)-poly(propylene oxide)-poly(ethylene oxide) triblock copolymer, which can be Pluronic F68 or Pluronic F127) through self-assembly and hydrogen bonds. An external magnetic field is applied, such that the temperature of the nano-magnetic particle rises up to a predetermined temperature range (about 37-50° C.) to give rise to a volume change of the drug carrier, a structural change or a destruction of the drug carrier, so as to continuously and slowly or quickly release the drug encapsulated in the composite polymer.

Preferably, for achieving an approaching-zero drug leakage and changing the release mode of the drug, the drug carrier of the present invention further comprises a shell for covering the surface of the composite polymer. The shell is made of an inorganic material selected from the group of silicon dioxide, titanium dioxide, and hydroxyapatite.

To achieve the aforementioned objective, the present invention further provides a use of the above-mentioned drug carrier with thermal sensitivity as an NMR contrast agent.

To achieve the aforementioned objective, the present invention further provides a manufacturing method of a drug carrier with thermal sensitivity, and the manufacturing method comprises the following steps. In an organic solvent, a nano-magnetic particle and a drug are mixed uniformly. A water-soluble polymer and a thermosensitive copolymer are dissolved into a water solution to self-assembly into a composite polymer. And then, the water solution containing the composite polymer and the organic solvent containing the drug and the nano-magnetic particle are mixed together and shaken to form an emulsion. Through self-assembly and hydrogen bonds and evaporating the organic solvent, the nano-magnetic particle and the drug are encapsulate into the composite polymer, so as to produce a stable drug carrier with an outer polymer layer.

Preferably, the manufacturing method of the drug carrier according to the present invention further comprises the following steps. The drug carrier manufactured according to the foregoing procedure is added into a mixed solution containing alcohol and another water solution, and then a silica-based precursor such as tetraethoxysilane (TEOS) is added into a mixed solution. By the hydrolysis and condensation of the silica-based precursor, the surface of the drug carrier is covered with an inorganic shell containing silicon (which is the aforementioned shell).

The drug carrier with thermal sensitivity, the manufacturing method thereof, and the use thereof have one or more of the following advantages.

(1) The shell made of an inorganic material is covered onto the surface of the drug carrier of the present invention, so that the effect of reducing drug leakage can be achieved before the drug encapsulated into the drug carrier arrives at a target position.

(2) The composite polymer of the present invention is made of a water-soluble polymer (such as poly vinyl alcohol) and a thermosensitive copolymer (such as Pluronic F68 or F127) in a specific proportion by a self-assembly without using any chemical crosslinking agent, so that the drug carrier of the present invention features a low toxicity.

(3) Because the composite polymer of the present invention is mixed from the water-soluble polymer and the thermosensitive copolymer in a specific proportion, the volume contraction/expansion ratio thereof exceeds 800.

(4) When the external magnetic field is applied, the temperature of the drug carrier of the present invention increases to 40-47° C. This will cause the drug carrier of the present invention to contract quickly, and the size thereof is reduced up to approximately 10 fold to give rise to a volume change of approximate 1000 fold.

(5) With the nano-magnetic particle (such as iron oxide) in the drug carrier of the present invention and the external magnetic field, the drug carrier of the present invention can be used as an NMR contrast agent for living organisms.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic view of the structure of a drug carrier with thermal sensitivity in accordance with an embodiment of the present invention;

FIG. 2 is a flow chart of a manufacturing method of a drug carrier with thermal sensitivity in accordance with a first embodiment of the present invention;

FIGS. 3A-3C are transmission electron microscope (TEM), scanning electron microscope (SEM) and high-resolution transmission electron microscope (HR-TEM) diagrams of a drug carrier manufactured in accordance with a second embodiment of the present invention respectively;

FIGS. 4A-4C shows the drug release behavior of different drug carriers while an external magnetic field is applied;

FIG. 5 is a graph showing the drug release behavior of a drug carrier-a to a drug carrier-d without a silica shell of the present invention at room temperature;

FIG. 6 is a graph showing the drug release behavior of a drug carrier-1 to a drug carrier-4 with a silica shell of the present invention at room temperature;

FIG. 7 is a histogram showing a change of particle diameter of the drug carrier-1 to the drug carrier-4 of the present invention at the temperature 40° C. and 47° C.;

FIG. 8 is a graph showing the drug release behavior of the drug carriers-1 to the drug carrier-4 of the present invention after being applied with an external magnetic field;

FIG. 9 is a TEM diagram of the drug carrier-1 of the present invention after being applied with an external magnetic field;

FIG. 10 is a confocal microscopic diagram of a drug carrier co-cultured with retinal pigment epithelium cells in accordance with the present invention;

FIG. 11 is a histogram showing the cell toxicity of a drug carrier of the present invention provided that no magnetic field is applied and a magnetic field is introduced for one minute;

FIG. 12A is a graph showing the particle diameter of a drug carrier-5 to a drug carrier-8 varied with temperature in accordance with the present invention;

FIG. 12B is a graph showing the drug release behavior of the drug carrier-6 after being applied with a magnetic field for 0, 15 and 30 seconds in accordance with the present invention;

FIG. 12C is a graph showing the drug release behavior of a drug carrier-7 after being applied with a magnetic field for 30 and 90 seconds in accordance with the present invention;

FIG. 13 shows graphs of different iron ion concentrations of a drug carrier of the present invention measured at Ti weighted and T2 weighted measurements respectively;

FIG. 14 shows NMR diagrams of a rat's brain before and after injecting a drug carrier of the present invention respectively; and

FIG. 15 shows NMR diagrams of a rat's liver and kidney before and after injecting a drug carrier of the present invention respectively.

### DESCRIPTION OF THE PREFERRED EMBODIMENTS

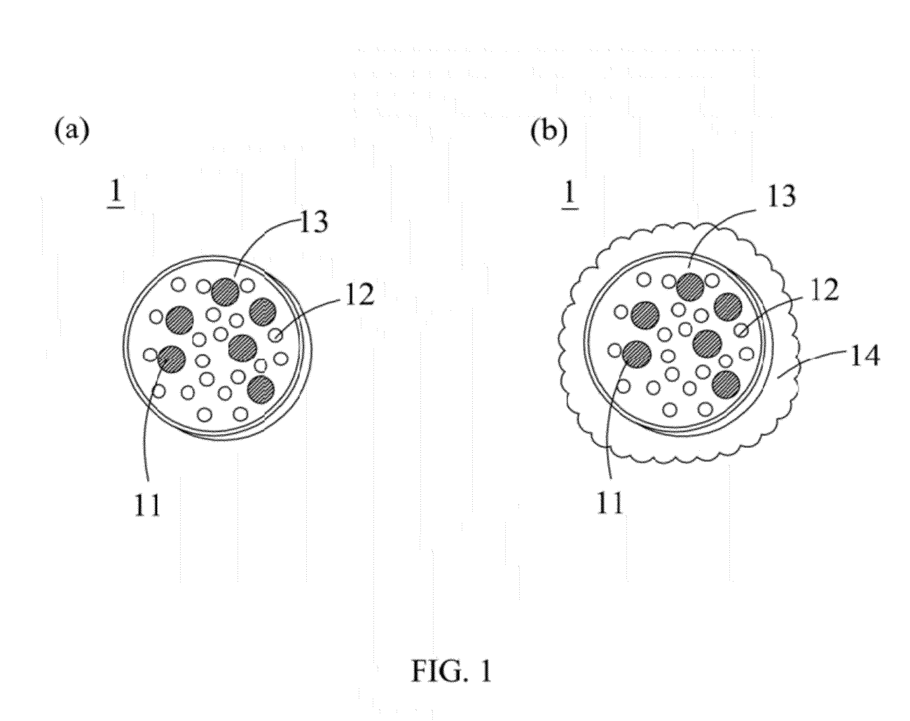

With reference to FIG. 1 for a schematic view of the structure of a drug carrier with thermal sensitivity in accordance with an embodiment of the present invention, FIG. 1(a) shows a drug carrier 1 of the present invention comprising a nano-magnetic particle 11, a drug 12 and a composite polymer 13. The nano-magnetic particle 11 and the drug 12 are encapsulated into the composite polymer 13. The composite polymer 13 is self-assembled by two kinds of polymers in a specific ratio, and these two polymers are a water-soluble polymer and a thermosensitive copolymer respectively. Preferably, the weight ratio of the water-soluble polymer to the thermosensitive copolymer is 1:10-10:1.

In a preferred embodiment, the drug carrier 1 of the present invention further comprises a shell 14 made of an inorganic material (such as silicon dioxide, titanium dioxide or hydroxyapatite), and the shell 14 is covered onto a surface of the composite polymer 13 as shown in FIG. 1(b). Thus, the drug carrier 1 of the present invention can achieve the effect of releasing the drug slowly.

Additionally, the drug carrier 1 is preferably in a spherical shape, but it also can be in any other shape. The drug carrier without the shell has a diameter between 10 nm to 500 nm; the nano-magnetic particle has a diameter between 3 nm to 30 nm; and the shell has a thickness between 1 nm to 50 nm.

When an external magnetic field is applied at a target position, the drug carrier 1 of the present invention can be moved to a target position due to the nano-magnetic particle 11 encapsulated into the drug carrier 1. The nano-magnetic particles 11 are applied with the external magnetic field, such that the nano-magnetic particles 11 produce heat, and the temperature of the nano-magnetic particles 11 rise to a predetermined temperature range (about 37-50° C.), causing a significant volume change, a structural change or a destruction of the drug carrier 1, so as to release the drug 12 in the composite polymer 13 quickly to a target position.

The aforementioned nano-magnetic particle 11 can be a nano-particle of ferrous oxide (Fe₂O₄), iron oxide (Fe₃O₄), cobalt iron oxide (CoFe₂O₄), manganese iron oxide (MnFe₂O₄) or manganese oxide. On the other hand, a precursor such as ferrous chloride (FeCl₂), ferric chloride (FeCl₃), cobalt chloride (CoCl₂), ferric nitrate (Fe(NO₃)₂), ferric acetate (Fe(CH₃COO)₃), cobalt acetate (Co(CH₃COO)₂), or manganese acetate (Mn(CH₃COO)₂) is used for producing the nano-magnetic particle 11. The water-soluble polymer includes poly vinyl alcohol (PVA), and the thermosensitive copolymer includes poly(ethylene oxide)-poly(propylene oxide)-poly(ethylene oxide) triblock copolymer (PEO-PPO-PEO polymer), poly(N-isopropyl acrylamide), gelatin or chitin. The poly(ethylene oxide)-poly(propylene oxide)-poly(ethylene oxide) triblock copolymer has the following structural formula.

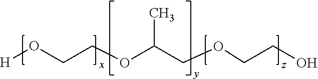

Where, if x=76, y=29, z=76, the poly(ethylene oxide)-poly(propylene oxide)-poly(ethylene oxide) triblock copolymer is Pluronic F68. If x=100, z=100, the poly(ethylene oxide)-poly(propylene oxide)-poly(ethylene oxide) triblock copolymer is Pluronic F127.

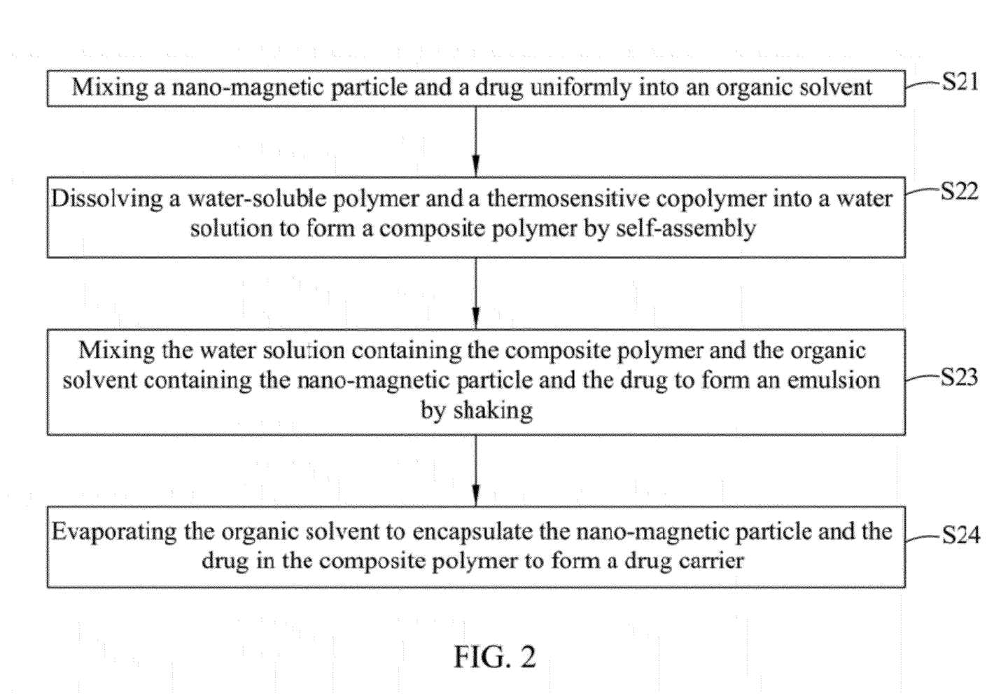

With reference to FIG. 2 for a flow chart of a manufacturing method of a drug carrier with thermal sensitivity in accordance with a first embodiment of the present invention, the manufacturing method comprises the following steps. As shown, in step S21, a nano-magnetic particle and a drug are mixed uniformly in an organic solvent. In step S22, a water-soluble polymer and a thermosensitive copolymer are dissolved in a water solution and self-assembled to produce a composite polymer. In step S23, the water solution containing the composite polymer and the organic solvent containing the nano-magnetic particle and the drug are mixed uniformly and shaken to form an emulsion. In step S24, the organic solvent is evaporated to encapsulate the nano-magnetic particle and the drug into the composite polymer to form a drug carrier.

To cover a shell onto a surface of the drug carrier, the following steps are carried out after the step S24. A mixed solution containing alcohol and another water solution is added into the drug carrier, and then a silica-based precursor, such as tetraethoxysilane (TEOS), is added to the foregoing mixed solution. The inorganic shell containing silicon (which is the shell as shown in FIG. 1) is covered onto the surface of the drug carrier by the hydrolysis and condensation of the silica-based precursor.

For making persons ordinarily skilled in the art understand the structure and effects of the drug carrier with thermal sensitivity according to the present invention, the following manufacturing procedure and function test of the drug carrier of the present invention are provided. It is noteworthy to point out that the following materials and parameters including concentration, content and response time are provided for illustrating the invention only. The invention is not limited to these arrangements only, but any equivalents can be adopted.

In a manufacturing method of a drug carrier with thermal sensitivity in accordance with a second embodiment of the present invention, the nano-magnetic particles are iron oxide (Fe₃O₄) nanoparticles, but the invention is not limited to such arrangement. Firstly, the iron oxide nano-particles (0.5 wt %) and the drug (0.1%) are dissolved into approximately 2 mL of chloroform. Pluronic F68 as a thermosensitive copolymer and poly vinyl alcohol (PVA) as a water-soluble polymer are heated at about 70° C. and dissolved in deionized water for approximately one hour until the deionized water presents clear. Then, the deionized water is cooled to room temperature. The deionized water and the chloroform containing the iron oxide nanoparticles and the drug are mixed, wherein the volume ratio of deionzied water to chloroform is about 5:2. After mixed, a strong ultrasonic vibration is performed for about 3 minutes to produce an emulsified solution. The emulsified solution is stirred at room temperature for 24 hours to evaporate all of the organic solvent. The iron oxide nanoparticles and drug without encapsulating into the composite polymer and remaining polymers are removed by centrifuge, and then rinsed by deionzied water for about three times to obtain a drug carrier of the present invention.

In addition, the shell is prepared by adding the drug carrier into a mixed solution containing alcohol and water, and then adding 1% of tetraethoxysilane (TEOS) into the mixed solution to perform hydrolysis and condensation. After about 30 minutes, ammonia water is added to accelerate the reaction. After the reaction for one day, a drug carrier with a silica shell can be collected. The collected drug carrier is rinsed by deionzied water for several times to remove any non-reacted substance.

The drug carrier with a highly temperature sensitivity in accordance with the present invention is prepared by mixing two kinds of polymers in a specific ratio, such that a novel polymer with high stability and super temperature sensitivity can be produced. The magnetic field can be used to drive the drug to release quickly, and a nano-material manufacturing process can be used for controlling the structure of the drug carrier can be controlled up to its best feature by the manufacturing process of the nano-materials.

Pluronic F68 has the thermosensitive property, but its structure is too loose and unable. The structure of Pluronic F68 may be damaged easily when the nano drug carrier is delivered into human body, thus resulting in a natural release of the drug. This situation is not good for the drug delivery system in the human body, so that the second kind of water-soluble polymer (poly vinyl alcohol) is doped to enhance the internal bonding of the nano carrier to produce a nano drug carrier with a high stability. In addition, a shell made of inorganic material covered onto the surface of the drug carrier can reduce the natural release effect of the drug significantly when no external magnetic field is applied.

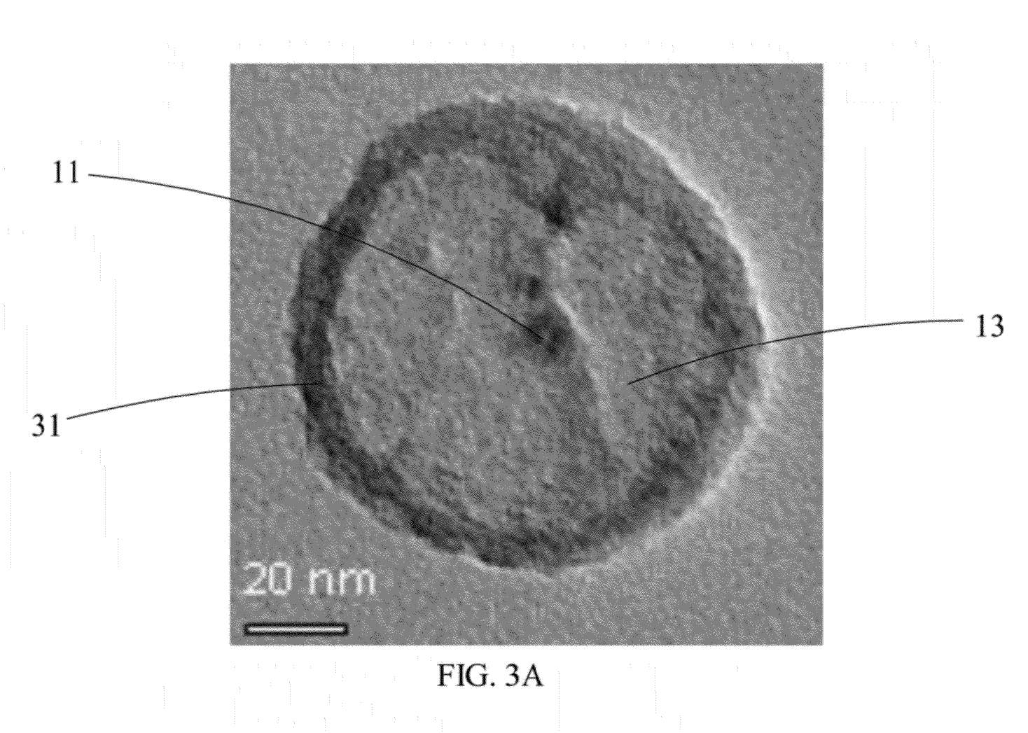

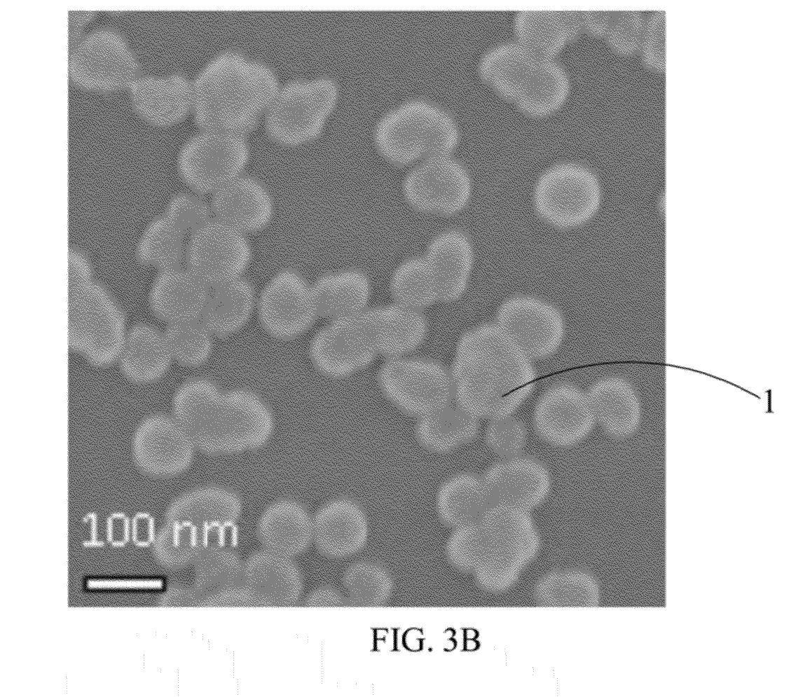

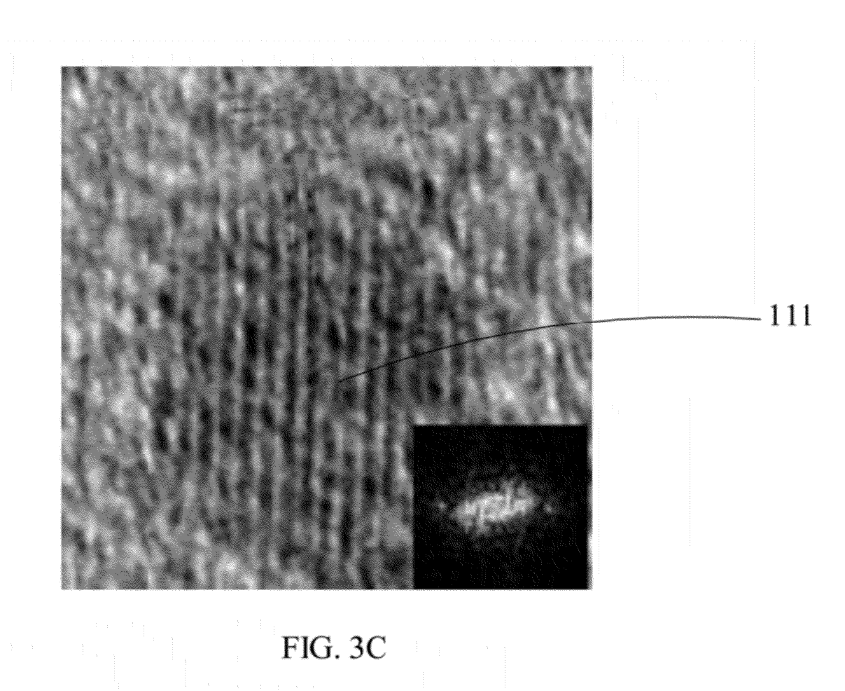

With reference to FIGS. 3A to 3C, FIGS. 3A to 3C illustrate transmission electron microscope (TEM), scanning electron microscope (SEM) and high-resolution transmission electron microscope (HR-TEM) diagrams of a drug carrier manufactured in accordance with the second embodiment of the present invention respectively. FIG. 3A shows the thickness of the silica shell 31 covered with the composite polymer 13 uniformly. The drug can be encapsulated into the composite polymer 13 easily by using a dense structure of silicon dioxide. In addition, the manufacturing process of this preferred embodiment can be conducted at room temperature for synthesizing a drug carrier without damaging the drug activity. In FIG. 3B, no crevice and crack can be observed form the surface of the drug carrier 1 of the present invention, and the shell 14 and the composite polymer 13 are integrated into a continuous structure without any gap between the two. The drug carrier 1 has an average diameter of 76 nm, and the silica shell 31 has a thickness of approximately 7 nm. In FIG. 3C, a crystal structure with iron oxide nanoparticles 111 can be observed clearly.

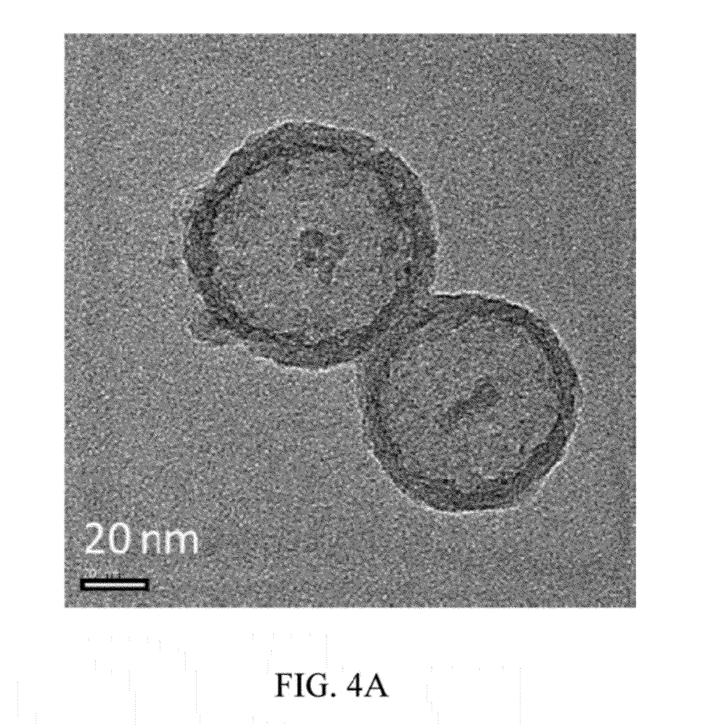

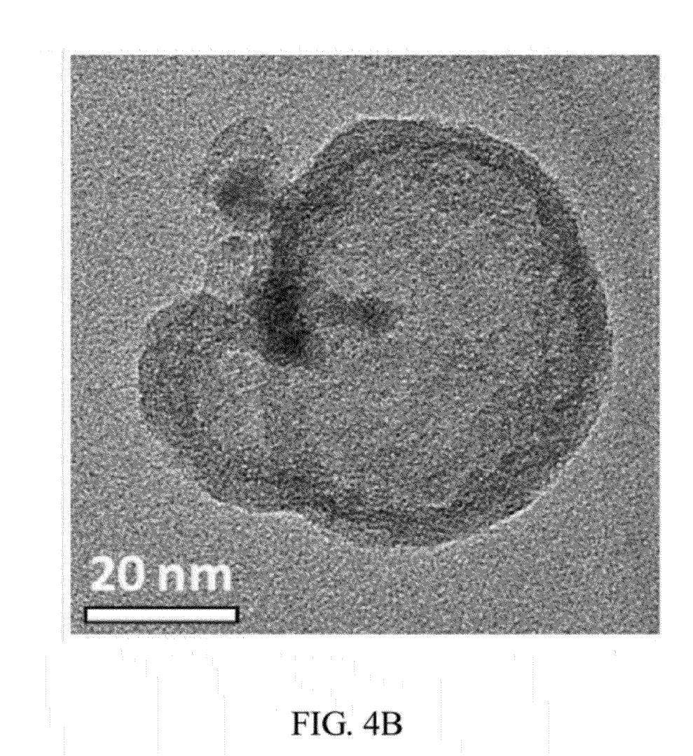

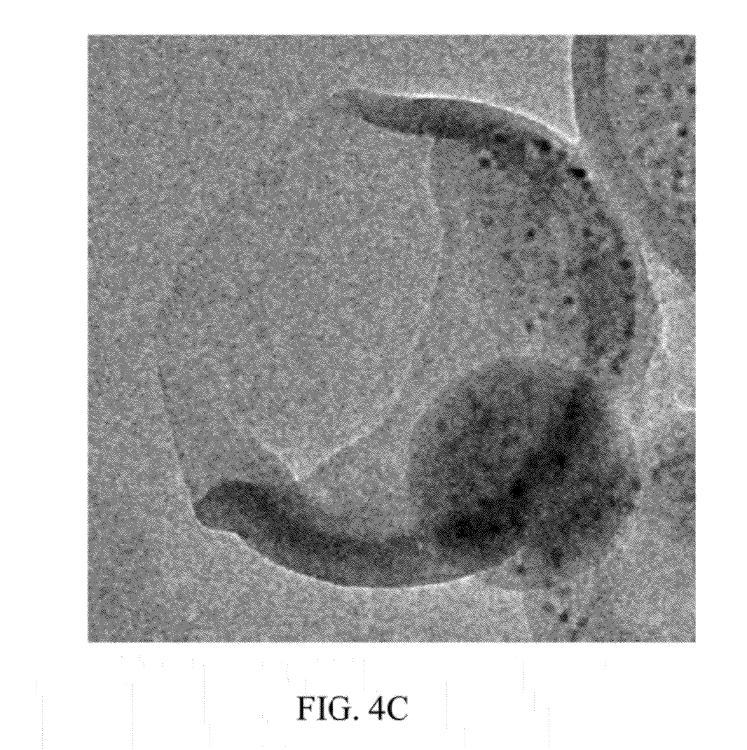

Furthermore, if the carrier does not have the thermosensitive copolymer (such as Pluronic F68), the structure of the carrier is still complete during applying the external magnetic field. When the proportion of the thermosensitive copolymer in the drug carrier is increased, the contraction level of the composite polymer comprised in the drug carrier is increased. Thus, the drug carrier with high proportion of the thermosensitive copolymer is collapsed and cracked quickly to achieve the rapid drug release. The results are shown in FIGS. 4A, 4B and 4C. FIG. 4A shows the drug carrier with PVA and the silica shell and without the Pluronic F68; FIG. 4B shows the drug carrier with Pluronic F68, PVA and the silica shell and the mixing ratio of the Pluronic F68 and PVA is 1/3; FIG. 4C shows the drug carrier with Pluronic F68, PVA and the silica shell, and the mixing ratio of the Pluronic F68 and PVA is 3/1. As shown in FIG. 4A, because there are not any thermosenitive copolymers (Pluronic F68) in the drug carrier, the drug carrier is not collapsed and cracked during applying the external magnetic field. However, when the thermosenitive copolymers (Pluronic F68) is added into the drug carrier and then the external magnetic field is applied, the drug carrier is collapsed and cracked so as to release the drugs. Additionally, the drug release of the drug carrier in FIG. 4C is faster than that of the drug carrier in FIG. 4B. Therefore, the collapse speed of the drug carrier depends on the proportion of the hermosensitive copolymer comprised in the drug carrier.

The manufacturing method of the second embodiment further adjusts the weight ratio of the water-soluble polymer to the thermosensitive copolymer, and the content, contraction level and drug release rate of the iron oxide nanoparticles are measured. The weight ratio of the water-soluble polymer to the thermosensitive copolymer and the content of the iron oxide nanoparticles of different drug carriers according to the present invention are listed in Table 1.

TABLE 1

| | | | | Organic/ | Saturated | | Encapsulation |
|---|---|---|---|---|---|---|---|
| Drug | Weight Ratio | | | Inorganic | Magnetization | Fe₃O₄ | Efficiency |
| Carrier | Fe₃O₄ | PVA/F68 | TEOS | (TGA) | (emu/g) | (%) | (%) |
| 1 | 1 | 5/1 | 2.5 | 66/34 | 7.9 | 12 | 73 ± 5 |
| 2 | 1 | 5/2.5 | 2.5 | 72/28 | 6.7 | 10 | 70 ± 4 |
| 3 | 1 | 5/5 | 2.5 | 79/21 | 4.9 | 8 | 65 ± 4 |
| 4 | 1 | 5/10 | 2.5 | 85/15 | 3.1 | 5 | 58 ± 5 |

Wherein, the encapsulation efficiency can be calculated by an equation of “(Total LBU Content−Remaining IBU Content in a Drug Carrier)/Total IBU Content×100”. The drug encapsulated in the drug carrier of the present invention is preferably a hydrophobic drug, and an analgesic anti-inflammatory ibuprofen (IBU) drug is used as a simulating drug in the present embodiment of the present invention.

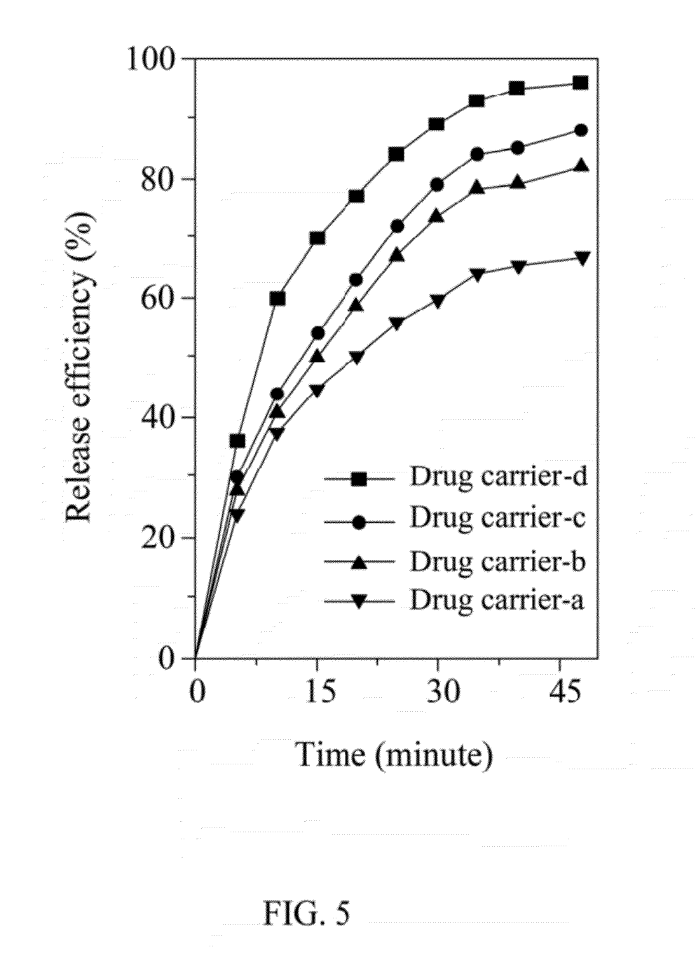

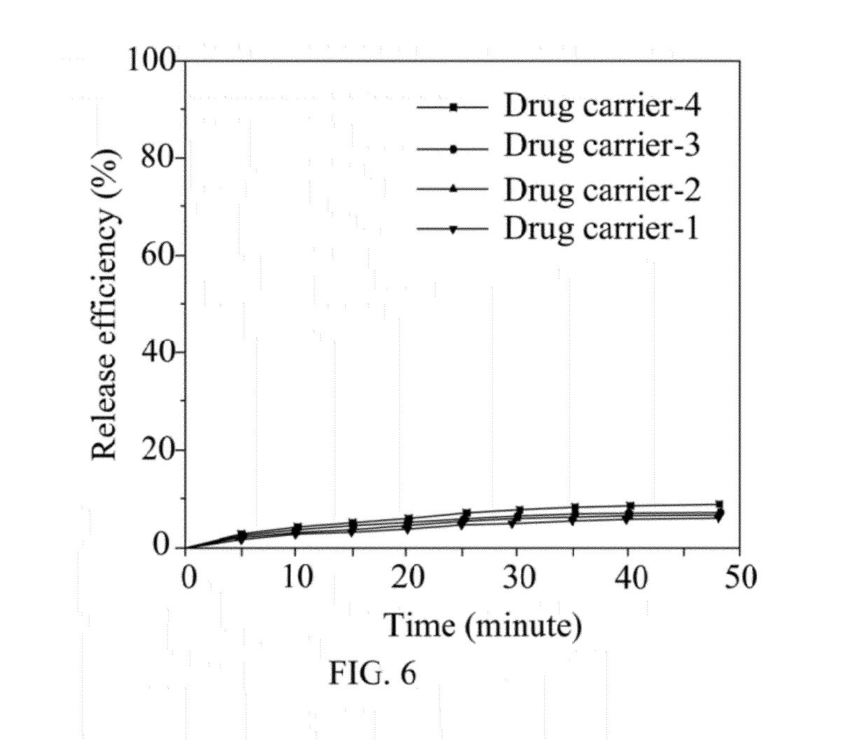

With reference to FIGS. 5 and 6, those are a graph showing the drug release behavior of a drug carrier-a to a drug carrier-d without a silica shell of the present invention at room temperature, and a graph showing the drug release behavior of a drug carrier-1 to a drug carrier-4 with a silica shell of the present invention at room temperature, respectively. As shown, the composite polymer for making the carriers is formed by polymerizing poly vinyl alcohol (PVA) and Pluronic F68, and the mixing ratios are 5/1, 5/2.5, 5/5 and 5/10 (as listed in Table 1).

In FIG. 5, the drug carrier-a to the drug carrier-d of the present invention are respectively the drug carrier-1 to the drug carrier-4 without the shell formed by TEOS. When the drug carrier of the present invention is not covered by the shell yet, the drug release speed is very fast. For example, over 60% of the drugs will be released in approximately 45 minutes.

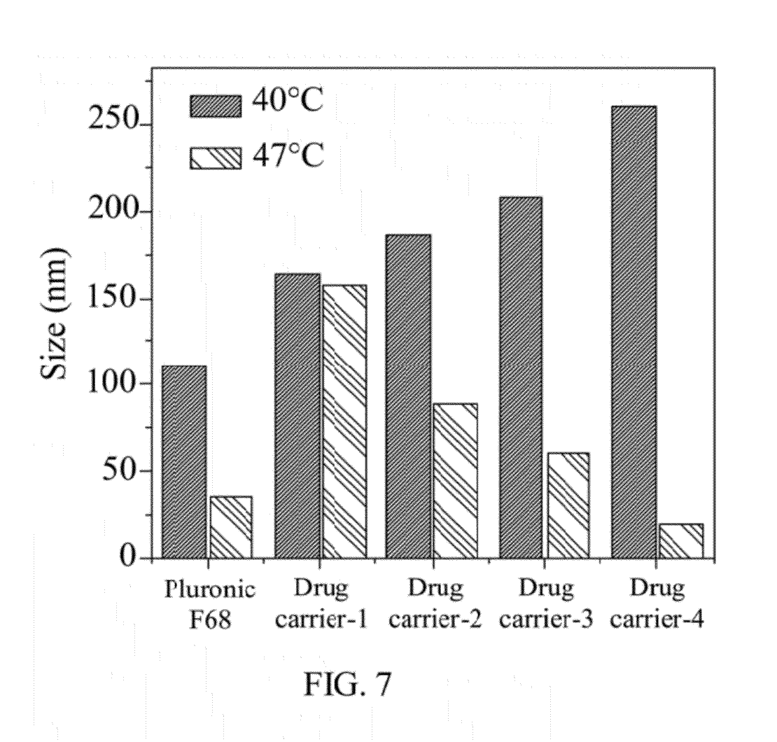

In FIG. 6, after the drug carrier-a to the drug carrier-d are encapsulated by the silica shell (which are the drug carrier-1 to the drug carrier-4 as shown in FIG. 1), only 5% of the drugs will be released naturally after 45 minutes. This is because poly vinyl alcohol is added into the drug carriers, such that the instability of the original drug carrier simply adopting Pluronic F68 can be improved. Most of the drugs are maintained in the composite polymer of the drug carriers 1-4 by covering the silica shell while transmitting, and the drug will not be released from the composite polymer until the drug carriers reach the target tissue. In the figure, although poly vinyl alcohol has no thermosensitivity, the poly vinyl alcohol can provide a large number of hydrogen bonds for stabilizing the structure. If the content of the poly vinyl alcohol is too low, the internal interaction of the drug carrier will not be strong enough to result that stable nano spherical structures cannot be formed easily. When the proportion of the poly vinyl alcohol is increased, a large number of hydrogen bonds can stabilize the nano structure of the drug carrier and help forming the silica shell to reduce the drug leakage. Therefore, the natural release rate of the drug carrier-1 is the lowest. That is, the drug encapsulation efficiency is the highest. With reference to FIG. 7, it is a histogram showing a change of particle diameter of the drug carrier-1 to the drug carrier-4 of the present invention at the temperature 40° C. and 47° C. The temperature sensitivity of the drug carrier of the present invention is very obvious under the interaction of poly vinyl alcohol and Pluronic F68, and the volume change at a specific temperature (which is 47° C. in this preferred embodiment) can even exceed 800 fold. In addition, the change of particle diameter of the drug carrier at different temperatures shows that the particle diameter of the drug carrier decreases instantaneously at the transition temperature of 47° C. If the ratio of Pluronic F68 to poly vinyl alcohol is controlled appropriately, the change of the particle diameter of the drug carrier can even reach up to 10 fold, and the volume change approaches 1000 fold. The drastic change of the particle diameter causes the silica shell to collapse and crack, so as to achieve the drug releasing effect.

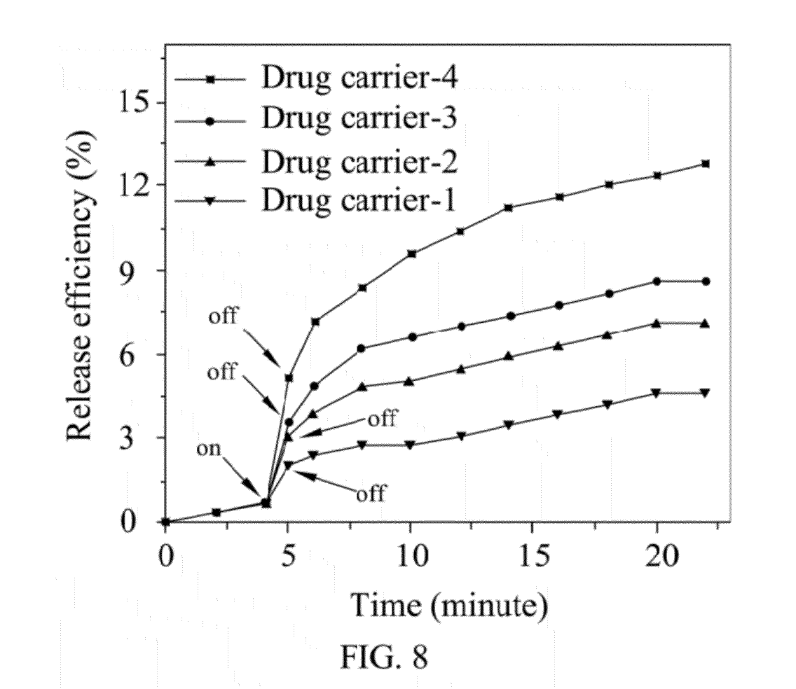

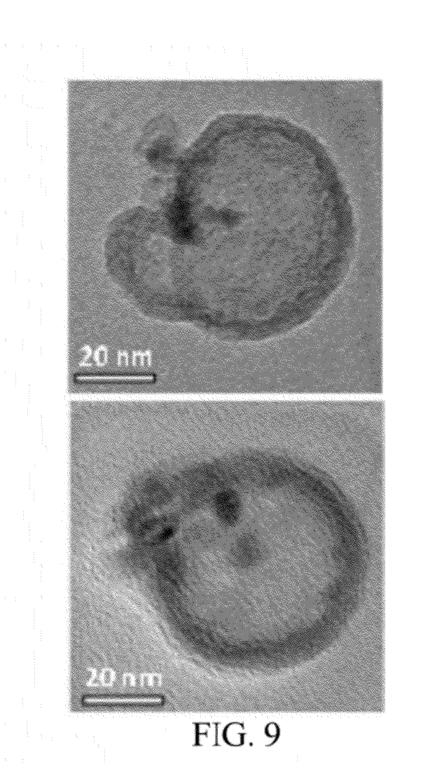

With reference to FIGS. 8 and 9, those are a graph showing the drug release behavior of the drug carrier-1 to the drug carrier-4 of the present invention after being applied with an external magnetic field and a TEM diagram of the drug carrier-1 of the present invention after being applied with an external magnetic field respectively. As shown, only a very small amount of the drugs is released before the external magnetic field is applied, and it shows that the drugs can be stored in the composite polymer. Until the external magnetic field is applied (approximately for 4.5 minutes, and indicated by “on” in the figure), and then is turn off (after applying the external magnetic field for approximately 30 seconds, and indicated by “off” in the figure), the IBU drugs are released from the composite polymer to the outside quickly as shown in FIG. 8. The result shows that the thermosensitive drug carrier of the present invention has excellent magnetic field sensitivity. After being applied with the magnetic field, the silica shell has significant cracked as shown in FIG. 9. The foregoing test illustrates that the thermosensitive drug carrier of the present invention can achieve the effect of releasing drugs through the external magnetic field.

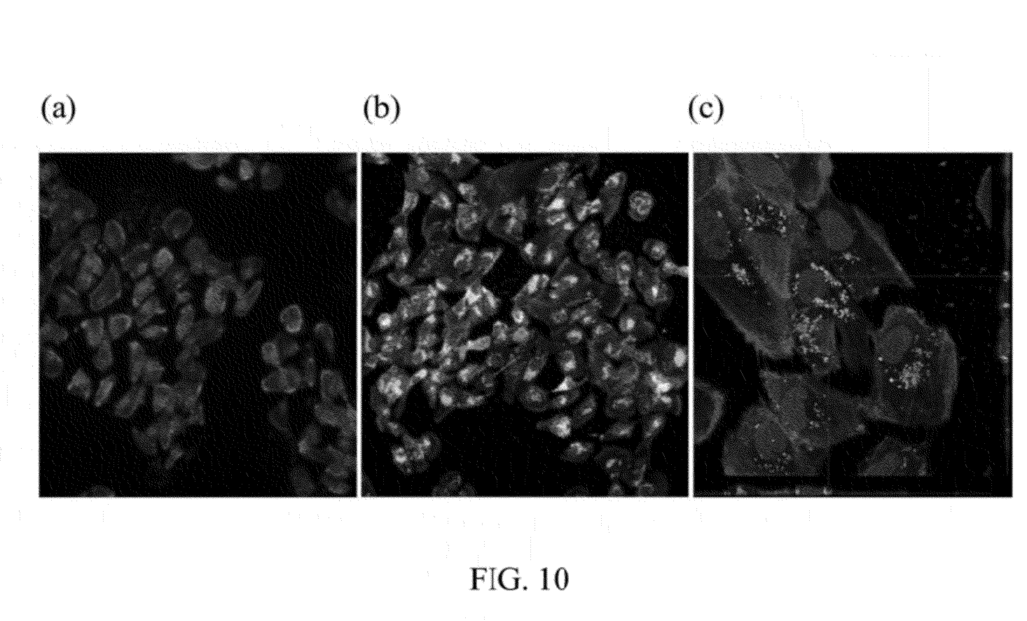

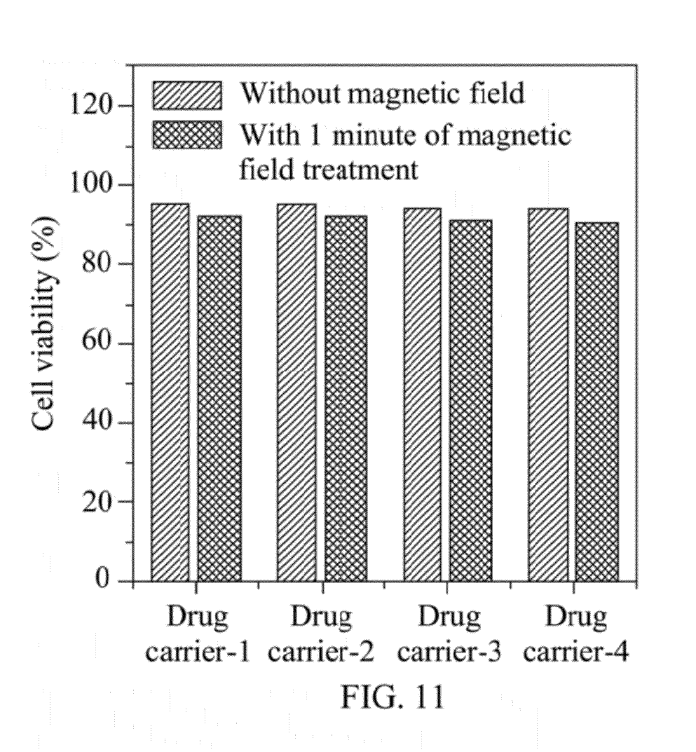

For testing the cell compatibility of the drug carrier of the present invention, the drug carrier of the present invention is marked with green fluorescence (FITC), co-cultured with retinal pigment epithelium cells (ARPE-19), and observed by a confocal microscope in the present embodiment. The results are shown in FIG. 10. After the drug carrier of the present invention and the cells are co-cultured for 4 hours, no significant uptake of the drug carrier by the cells is found (showing a very small quantity of green spots) as shown in FIG. (a). However, if the culturing time is increased to 24 hours, a large quantity of green spots can be found in the cells as shown in FIGS. (b) and (c), indicating that the drug carriers are entered into the cytoplasm of the cells and the cells still maintain the size and shape. The data above show that the drug carriers of the present invention have good cell compatibility. In the cytotoxicity assay, the cell survival rate is over 90% under the condition of applying the magnetic field and culturing for a long time as shown in FIG. 11.

In another preferred embodiment, the thermosensitive copolymer Pluronic F127 is used to substitute the Pluronic F68 of the previous preferred embodiment, and the weight ratio of iron oxide nanoparticle to poly vinyl alcohol/Pluronic F127 and the critical transition temperature are listed in Table 2 as follows.

TABLE 2

| Drug carrier of | | | |
|---|---|---|---|
| the present | Weight Ratio | | Critical Transition Temperature |
| invention | Fe₃O₄ | PVA/F127 | (° C.) |
| 5 | 1 | 1/4 | 32.7 |
| 6 | 1 | 2/3 | 38.0 |
| 7 | 1 | 3/2 | 42.6 |
| 8 | 1 | 4/1 | 45.4 |

From Table 2, the PVA/F127 in accordance with this preferred embodiment of the present invention is a composite polymer with a ratio of 1/4, 2/3, 3/2 and 4/1.

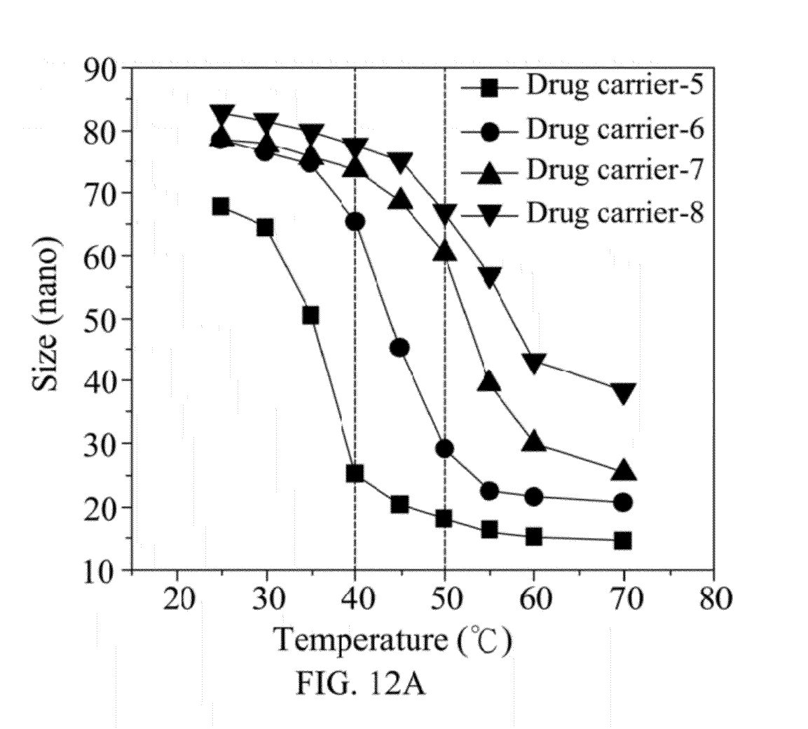

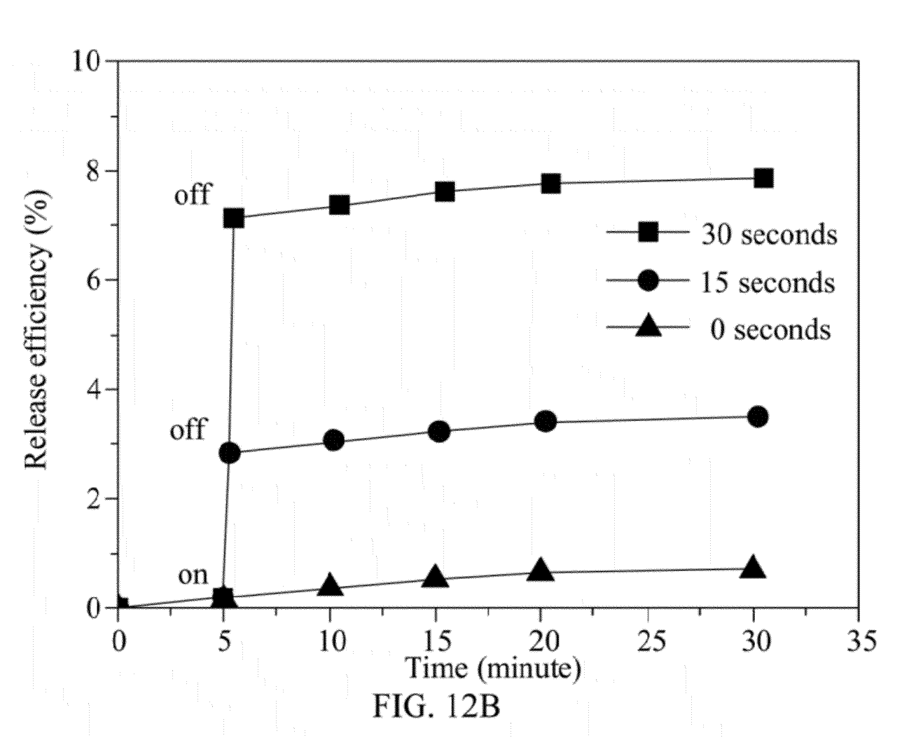

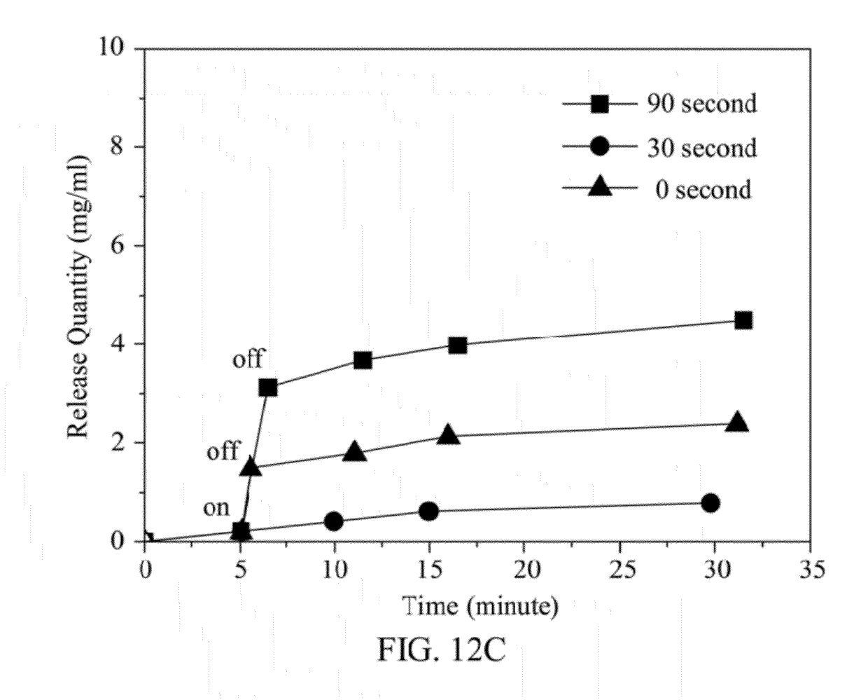

With reference to FIG. 12A, it is a graph showing the particle diameter of a drug carrier-5 to a drug carrier-8 varied with temperature in accordance with the present invention. The drug carrier-5 to the drug carrier-8 have the largest volume change rate at a temperature range of 37-50° C., and the drug carrier of the present invention becomes smaller quickly as the temperature changes. Since human body temperature is about 37-39° C., the drug carrier of the present embodiment can maintain a stable structure without releasing much drugs naturally when the drug is transmitted to a specific position of a human body by applying the magnetic field. However, if Pluronic F127 is only used for manufacturing the drug carrier of the present invention, the critical transition temperature is lower than others (about 25.8° C. which is not listed in the table). If only poly vinyl alcohol is used as the drug carrier of the present invention, there is no critical transition temperature at all.

Thus, just the poly vinyl alcohol or only Pluronic F127 is not suitable for the manufacture of a thermosensitive drug carrier.

With reference to FIG. 12B, it is a graph showing the drug release behavior of the drug carrier-6 after being applied with a magnetic field for 0, 15 and 30 seconds in accordance with the present invention. As shown, the external magnetic field is applied to the drug carrier-5 of this preferred embodiment at the fifth minute, and lots of drugs are released by applying the magnetic field not only for 15 seconds but also 30 seconds. The quantity of released drugs is related to the time duration of applying the magnetic field. In other words, the longer the applied magnetic field, the larger the quantity of the released drugs. If the drug carrier-7 having high contents of poly vinyl alcohol (PVA/Pluronic F127=3/2) can be used to perform the drug release test, a lower and slower drug release is found as shown in FIG. 12C. The results indicate that the drug releasing behavior of the drug carrier of the present invention can be controlled by modifying the contents of the composition.

In addition, the drug carrier of the present invention can be used as an NMR contrast agent, and the following animal experiment is performed. As the medical applications and development of the NMR technology advance, NMR becomes a representative image processing method for testing brains, spines, muscles and bones. The NMR imaging technology is mainly based on signals measured from hydrogen atoms in water of each tissue, and such signals can be divided into two vectors in different directions based on coordinates. These two vectors include a longitudinal relaxation (T1) and a transverse relaxation (T2), and the mechanisms of these two signals are different and will not affect one another.

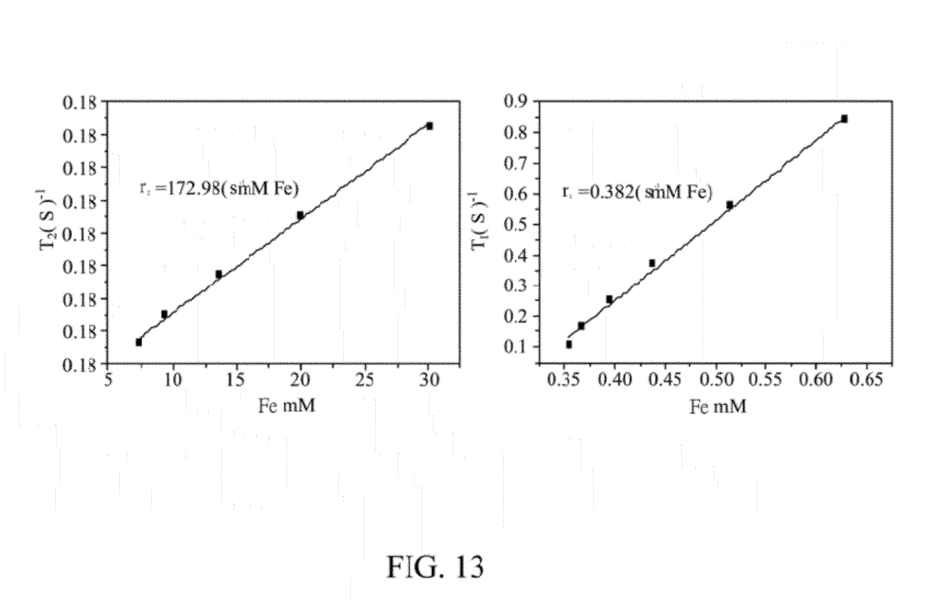

A general NMR contrast agent adopting the T1 mechanism comprises ions with the high paramagnetic property. Most commonly, a small organic chelating agent is reacted with gadolinium ions to obtain a stable organic metal complex. The nano-magnetic particle has a diameter falling within a range of 3 to 10 nano, such that the nano-magnetic particle can be developed and applied to the NMR contrast agent. In most cases, these magnetic particles adopt the T2 mechanism to affect the result of the image. With reference to FIG. 13 for graphs of different iron ion concentrations of a drug carrier of the present invention measured at T1 weighted and T2 weighted measurements respectively, the drug carrier has the properties of the T2 contrast agent. In addition, the spin-spin relaxation rate (r₂) of the drug carrier of the present invention falls within the range of 10s⁻¹M⁻¹<r₂<500s⁻¹M⁻¹.

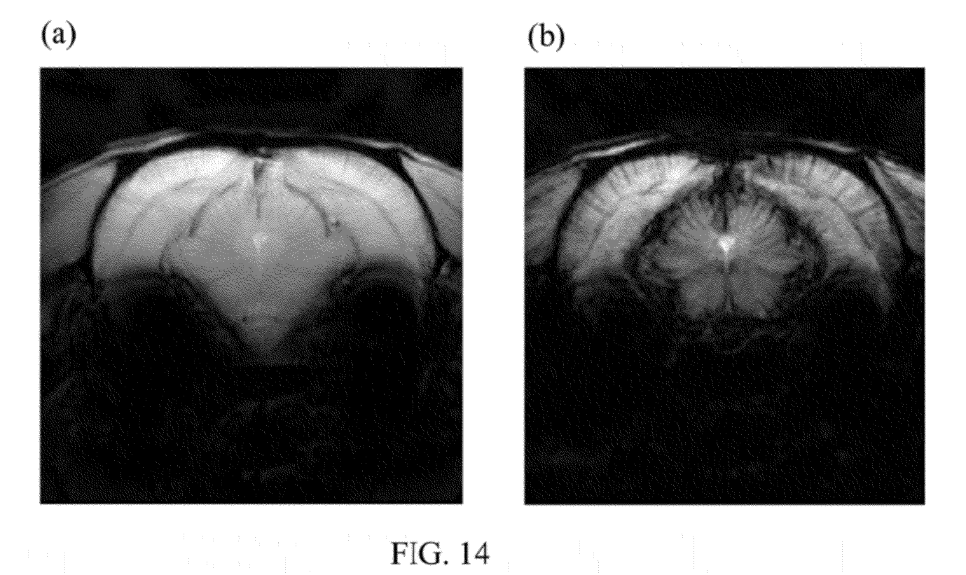

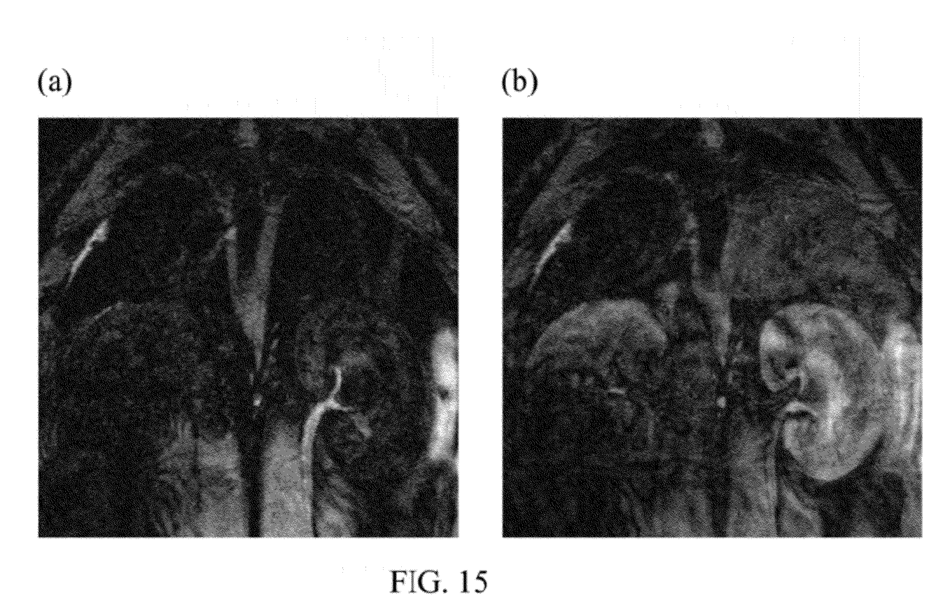

Furthermore, after the drug carrier of the present invention is injected into a rat, the rat's brain vascular contrast increases significantly as shown in FIG. 14. In FIG. 14, FIG. (a) shows that the drug carrier of the present invention has not been injected, and FIG. (b) shows that the drug carrier of the present invention has been injected. With reference to FIG. 14 for NMR diagrams after the drug carrier of the present invention is injected into a rat's body such as its liver and kidney. In FIG. 15, FIG. (a) shows an NMR image of the rat's liver and kidney before the drug carrier of the present invention is injected into the rat, and FIG. (b) shows an NMR image of the rat's liver and kidney after the drug carrier of the present invention is injected into the rat. From the above results, the contrast is increased significantly after injecting the drug carrier of the present invention.

In the results as shown in FIGS. 13 to 15, the drug carrier and the location of a pathological tissue can be monitored through NMR imaging related technologies. When the drug carrier of the present invention enters into a human or animal body, the aforementioned method applying an external magnetic field at a target position (such as a liver, kidney, brain or spleen) can induce the drug carrier of the present invention to release the drug encapsulated in the composite polymer because of the magnetic force being the force at a distance, so as to achieve an effective treatment. The above data can show that the drug carrier of the present invention can release drug at a specific target effectively.

In addition, the surface of the shell can be modified with at least one biological molecule, and such biological molecule includes ribonucleic acid (RNA), deoxyribonucleic acid (DNA), protein, hapten, avidin, streptavidin, neutravidin, lectin or selectin. After the drug carrier of the present invention is coupled with biological molecules, the drug carrier can be used for targeting a tumor.

In summation of the description above, the composite polymer is formed by self assembling the water-soluble polymer (such as poly vinyl alcohol) and the thermosensitive copolymer (such as Pluronic F68 or F127) according to a specific ratio, and a simple and easy manufacturing process is adopted without using any chemical crosslinking agent. Therefore, the composite polymer is highly stable and thermosensitivity, and has a high volume change rate, a high biocompatibility and a low toxicity. In addition, the drug carrier of the present invention can improve the drug encapsulation efficiency, controllability and quick release, and even can be used as a NMR contrast agent to achieve the effects of tracking and positioning the position of the drug carrier in human or animal bodies and releasing the drug to a specific target.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A drug carrier with thermal sensitivity, comprising:
a nano-magnetic particle;
a drug; and
a composite polymer, formed by self-assembling a water-soluble polymer and a thermosensitive copolymer, and provided for encapsulating the nano-magnetic particle and the drug therein;
wherein, an external magnetic field is applied to rise the temperature of the nano-magnetic particle to a predetermined temperature range to cause a volume change of the drug carrier and a structural change or destruction of the drug carrier so as to release the drug encapsulated in the composite polymer quickly.

2. The drug carrier of claim 1, further comprising a shell covered onto a surface of the composite polymer.

3. The drug carrier of claim 2, wherein the shell is made of an inorganic material selected from the group of silicon dioxide, titanium dioxide and hydroxyapatite.

4. The drug carrier of claim 1, wherein the water-soluble polymer and the thermosensitive copolymer have a weight ratio of 1:10-10:1.

5. The drug carrier of claim 1, wherein the water-soluble polymer comprises poly vinyl alcohol (PVA).

6. The drug carrier of claim 5, wherein the thermosensitive copolymer is one selected from the group of poly(ethylene oxide)-polypropylene oxide)-poly(ethylene oxide) triblock copolymer, poly(N-isopropyl acrylamide), gelatin and chitin.

7. The drug carrier of claim 6, wherein the external magnetic field causes a volume contraction of the drug carrier to destruct the structure of the drug carrier.

8. The drug carrier of claim 1, wherein the nano-magnetic particle is a nano-particle selected from the group of ferrous oxide, iron oxide, cobalt iron oxide, manganese iron oxide and manganese oxide.

9. The drug carrier of claim 1, wherein the predetermined temperature range falls within 37-55° C.

10. A use of the drug carrier with thermal sensitivity according to claim 1 as a nuclear magnetic resonance (NMR) contrast agent.

11. A manufacturing method of a drug carrier with thermal sensitivity, comprising the steps of:
mixing a nano-magnetic particle and a drug uniformly into an organic solvent;
dissolving a water-soluble polymer and a thermosensitive copolymer into a water solution to form a composite polymer by self-assembly;
mixing the water solution containing the composite polymer and the organic solvent containing the nano-magnetic particle and the drug to form an emulsion by shaking; and
evaporating the organic solvent to encapsulate the nano-magnetic particle and the drug in the composite polymer to form a drug carrier.

12. The manufacturing method of a drug carrier with thermal sensitivity according to claim 11, further comprising the steps of:
adding the drug carrier into a mixed solution containing alcohol and another water solution;
adding a silica-based precursor into the mixed solution; and
performing a hydrolysis and a condensation of the silica-based precursor, to cover an inorganic shell containing silicon onto a surface of the drug carrier.

13. The manufacturing method of a drug carrier with thermal sensitivity according to claim 12, wherein the silica-based precursor includes tetraethoxysilane (TEOS).

14. The manufacturing method of a drug carrier with thermal sensitivity according to claim 11, wherein the water-soluble polymer and the thermosensitive copolymer have a weight ratio of 1:10-10:1.

15. The manufacturing method of a drug carrier with thermal sensitivity according to claim 11, wherein the water-soluble polymer comprises poly vinyl alcohol (PVA).

16. The manufacturing method of a drug carrier with thermal sensitivity according to claim 15, wherein the thermosensitive copolymer is one selected from the group of poly(ethylene oxide)-poly(propylene oxide)-poly(ethylene oxide) triblock copolymer, poly(N-isopropyl acrylamide), gelatin and chitin.

17. The manufacturing method of a drug carrier with thermal sensitivity according to claim 11, wherein the nano-magnetic particle is a nanoparticle selected from the group of ferrous oxide, iron oxide, cobalt iron oxide, manganese iron oxide and manganese oxide.
